(19) United States
(12) Patent Application Publication
(10) Pub. No.: US 2014/0005554 A1
(43) Pub. Date: Jan. 2, 2014

(54) Telemetric Sensing of Blood Flow Rates by Passive Infrared Temperature Sensing

(71) Applicants: Biswajit Das, Henderson, NV (US); Frank van Breukelen, Las Vegas, NV (US)

(72) Inventors: Biswajit Das, Henderson, NV (US); Frank van Breukelen, Las Vegas, NV (US)

(21) Appl. No.: 13/719,729

(22) Filed: Dec. 19, 2012

Related U.S. Application Data
(60) Provisional application No. 61/581,446, filed on Dec. 29, 2011.

(51) Int. Cl. A61B 5/026

(52) U.S. Cl. CPC A61B 5/0261
USPC 600/474

(57) ABSTRACT

Systems enable a method of determining the rate of flow of a bodily fluid through a vessel in a live patient by:
positioning at least two separate passive infrared sensors at two points along a length of the vessel;
sensing local temperature changes at the two points along the vessel to generate signals of the local temperature changes from each passive infrared sensor;
a processor receiving the signals and quantifying the local temperature changes at the two points; and
the processor executing code to determine blood flow rate in the vessel from the local temperature changes quantified by the processor at the two points.

### RELATED APPLICATION DATA

The present application claims priority from U.S. Provisional Patent Application 61/581,446 filed Dec. 29, 2011.

### BACKGROUND OF THE INVENTION

1. Field of the Invention

The present invention relates to the field of blood flow sensing, particularly telemetric monitoring of blood flow sensing, and more particularly with the use of low energy consumption devices in telemetric monitoring of blood flow sensing.

2. Background of the Art

Heart disease and stroke are the leading causes of death in Americans today. Stroke alone afflicts nearly 800,000 Americans per year and results in approximately 145,000 deaths (Strokecenter.org; Rogers et al., Rogers V L, Go A S, Lloyd-Jones D M, Adams R J, Berry J D, Brown T M, Carnethon M R, Dai S, de Simone G, Ford ES, Fox C S, Fullerton H J, Gillespie C, Greenlund KJ, Hailpern S M, Heit J A, Ho P M, Howard V J, Kissela B M, Kittner S J, Lackland D T, Lichtman J H, Lisabeth L D, Makuc D M, Marcus G M, Marelli A, Matchar D B, McDermott M M, Meigs J B, Moy C S, Mozaffarian D, Mussolino M E, Nichol G, Paynter N P, Rosamond WD, Sorlie P D, Stafford R S, Turan T N, Turner M B, Wong N D, Wylie-Rosett J (2011) Heart disease and stroke statistics—2011 Update: A report from the American Heart Association. Circ. 123:e18-e209.)

Estimated annual costs for the treatment of the consequences of stroke are approximately $40.9 billion. Despite the tremendous human and financial cost, current research models have been unable to lead to the development of a definitive therapy for the treatment of stroke. The majority of strokes involve impaired blood flow. It is believed that an inability to readily determine blood flow rate in unrestrained animals or for extended periods of time has hampered efforts at therapeutic developments.

Current strategies for measurement of blood flow rate include laser Doppler, ultrasonic, or electromagnetic methods of detection and/or the use of microspheres (Tabrizchi and Pugsley, Tabrizchi R, Pugsley M K. (2000) Methods of blood flow measurement in the arterial circulatory system. Journal of Pharmacological and Toxicological Methods 44:375-384.). While very effective, these cumbersome strategies consume large amounts of power which makes their application to telemetry virtually impossible.

Published U.S. Patent Application Document No. 20090292214 discloses Systems and methods for obtaining and acting upon information indicative of circulatory health and related phenomena in human beings or other subjects.

Published U.S. Patent Application Document No. 20110213217 describes an energy efficient wireless medical sensor that may be capable of optimizing battery life and increasing component life by selectively using only a subset of the sensors and sensor functionality included in the wireless medical sensor at any one time. One or more update factors may be used by the wireless sensor or an external patient monitor to derive a data collection modality, data collection rates, and update interval. The data collection modality, data collection rates, and update interval may be used to selectively gather sensing data in a manner that is more energy efficient.

Published U.S. Patent Application Document No. 20110118561 discloses a physiological monitoring system that can independently control multiple displays to provide displays of measured physiological parameters than can differ from each other in format and/or selected parameters. Individual display monitors can be customized to display the parameters of interest to a particular medical professional more prominently. In order to facilitate controlling multiple displays, a controller in communication with the physiological monitoring system can be attached or positioned near a user of a display. The controller can remotely change the display output from the physiological monitoring system. The controller can be attached to a particular display and control the corresponding output for that display. Typically, commands from the controller affect only the display output for the particular display and not the display output for other displays.

Published U.S. Patent Application Document No. 20110066042 describes an electronic monitoring device that includes an electronic processor (520) having at least one signal input for body monitoring, and a memory (530) holding instructions for the electronic processor coupled to the electronic processor so that the electronic processor is operable to isolate a cardiac signal including cardiac pulses combined with other cardiac signal variations, and the electronic processor further operable to execute a filter (730) that separates a varying blood flow signal from the cardiac pulses and to output information (790) based on at least the varying blood flow signal. Other devices, sensor assemblies, electronic circuit units, and processes are also disclosed.

Published U.S. Patent Application Document No. 20100298683 Devices and methods are described for wirelessly monitoring an emergency responder. In some embodiments, a sensor acquires values of carboxyhemoglobin in blood. The values are recorded and are used to provide feedback to a user. The feedback includes at least one of visible, tactile, and audible information.

Published U.S. Patent Application Document No. 20100130880 A system for detecting blood flow in the prostate comprises a blood flow sensor disposed on a catheter that can be inserted into a subject's urethra so that the blood flow sensor is located to detect blood flow in the subject's prostate gland. The sensor may be a sensor of a near infrared spectroscopy (NIRS) system configured to detect in the prostate one or more biocompounds indicative of blood flow. An output of the sensor may provide an input to a controller for a heater disposed to heat tissues of the prostate. Some embodiments comprise one or more additional sensors for detecting blood flow and/or temperature of a portion of the subject's rectal wall adjacent to the prostate gland. In such embodiments, outputs from the additional sensors may provide additional inputs to the controller.

Published U.S. Patent Applications Publication No. 20050184869 and 20040140430 (Micko) discloses designs enabling manufacture of efficient passive infrared motion sensors.

All patent applications and patents and literature cited herein is incorporated by reference in their entirety.

### BRIEF DESCRIPTION OF THE INVENTION

The external positioning of two passive infrared sensor (PIR) detectors at a fixed distance and longitudinally on a blood vessel will detect small temperature fluctuations within the flow stream; analysis of phase shifts of these small temperature fluctuations would allow for the precise determination of flow within the vessel. This system would allow for passive measurement of blood flow rate. This sensor is readily adaptable to wireless telemetric solutions and allows long term measurement of blood flow without the confounding effects of anesthesia, physical restraint, tethering or stress induced by individually housing social animals like rats.

### BRIEF DESCRIPTION OF THE FIGURES

FIG. 1 shows prior art evidence of changes in blood temperature as a function of time in a dog.

FIG. 2 shows a graph and diagram of sensor orientation according to the present invention.

FIG. 3 shows a structure of one embodiment of elements useful in the practice of the present technology.

### DETAILED DESCRIPTION OF THE INVENTION

Previous efforts to apply blood flow sensors to telemetry have been largely unsuccessful due to the inherent power consumption of the sensors. Most efforts have relied on reducing the power utilization of pre-existing technologies; few efforts have focused on new sensor development. We also note that there are no commercially available blood flow rate telemeters appropriate for small animal models like rats and mice.

The present system is innovative, yet relies on an underlying proven technology that has been exploited for years by the home security industry. Commercially available passive infrared (PIR) motion sensors rely on the detection of miniscule changes in temperature or heat. Certain crystalline materials have the property to generate a surface electric charge when exposed to thermal infrared radiation. This phenomenon is known as pyroelectricity. The passive infrared sensor module works on the same principle. The human body radiates heat in the form of infrared radiation which is maximum at about 9.4 μm. The presence of a human body creates a sudden change in the IR profile of the surrounding that is sensed by the pyroelectric sensor. The PIR-based motion sensor module has an instrumentation circuit on board that amplifies this signal to appropriate voltage level to indicate the detection of motion.

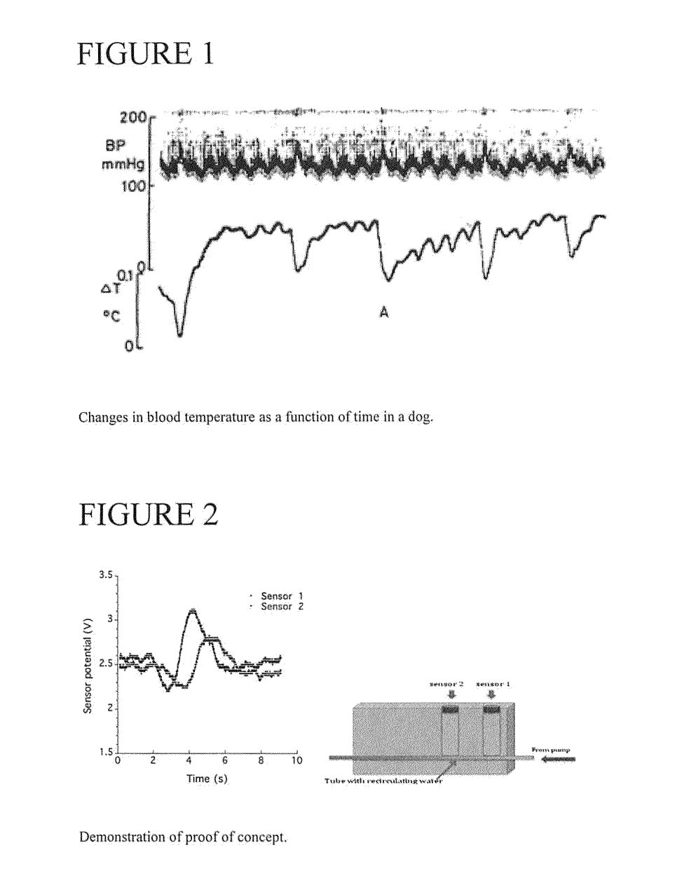

Although effective at detecting heat changes at distances of over 30 m, these sensors are passive in nature. No significant power needs to be supplied to the sensor. Examples of commercially available underlying sub-component technology which, upon exposure to infrared radiation, include lithium tantalate (LiTaO₃) or similar pyroelectric materials that produce an electrical potential. The positioning of two PIR detectors, placed at a fixed distance from each other within the flow stream or adjacent the flow stream enables an analysis of phase shifts of small temperature fluctuations that would allow for the precise determination of flow. Since respiratory rates (e.g., 12 breaths per minute for humans) are much lower than heart rates (e.g., 60 beats per min for humans) and there are changes in blood pressure due to the pulsatile nature of blood flow, it follows that there will be changes in temperature in flowing blood. Indeed, FIG. 1 demonstrates such changes in a dog. Effective application of PIR detectors allows exploitation of these seemingly small changes in temperature.

One approach to measuring flow rate via telemetry was offered by Bork et al. (Bork T, Hogg A, Lempen M, Müller D, Joss D, Bardyn T, Büchler P, Keppner H, Braun S, Tardy Y, Burger J. (2010) Development and in-vitro characterization of an implantable flow sensing transducer for hydrocephalus. Biomedical Microdevices 12:607-618.) These investigators utilized a thermal anemometer transducer to measure flow in artificial cerebrospinal fluid (CSF). A thermal anemometer uses a heated probe element in a fluid stream. Flow can then be inferred from the heating power necessary to maintain the probe at an elevated temperature. This power is proportional to flow rate. The system proved effective at estimating flow rate. However, and although significant efforts were made to reduce power consumption, the sensor alone consumes approximately 20 mW. A typical battery such as an LR44 watch battery (oftentimes used in telemetry) would only last ˜15 h with this system. Not surprisingly, the researchers employed a radio frequency based recharging system wherein the battery was charged inside of the animal. Disturbingly, the amount of power required to effectively sustain the telemeter resulted in significant warming (−2° C.). Furthermore, blood cells introduced into the CSF result in an altered thermal profile and limit the use of a thermal anemometer. Similarly, Yonezawa et al. (Yonezawa Y, Caldwell W M, Schadt J C, Hahn A W. (1989) A miniaturized ultrasonic flowmeter and telemetry transmitter for chronic animal blood flow measurements. Biomedical and Scientific Instrumentation 25:107-111.) exploited a miniaturized ultrasonic flowmeter to measure blood flow. Power consumption was 48 mW. These systems illustrate that despite major engineering efforts to minimize power use, a major barrier to chronic blood flow rate measurement has been a reliance on technologies that consume relatively large amounts of power. It is an element of the practice of the present technology that the availability of a passive sensor for blood flow rate, which requires no external power for operation, could make application to telemetry more successful.

FIG. 1 displays graphed changes in blood temperature as a function of time in a dog. Appelbaum et al. (Appelbaum A, Mahler Y, Nitzan M. (1982) Correlation of blood temperature fluctuations with blood pressure waves. Basic Research in Cardiology. 77:93-99.) employed amplified thermocouples to directly measure changes in blood temperature as it relates to blood pressure. An increase of blood pressure was correlated with an increase in the blood temperature in the pulmonary artery but a decrease in the blood temperature in the venae cavae. The changes in blood temperature are well within detection limits for a PIR sensor. In our application, any discernable change (increase or decrease) in temperature can be used for comparison between the two sensors to calculate flow rate. The entire trace is approximately 105 seconds. FIG. 1 is incorporated from Appelbaum et al., 1982.

One aspect of the present technology is to provide a miniature sensor that can be mounted on a blood vessel in a live animal and integrated to a low power telemetry system. A significant attribute to the system can be use of a sensor that would be small enough to be mounted on a blood vessel. The sensitivity of the sensor would need to be sufficient to detect small changes in blood temperature. As indicated above, PIR sensors are extremely sensitive as would be required to detect motion tens of meters away in air. For instance, lithium tantalite has a pyroelectric constant of 2.3×10⁴ C/m²·x° C. (http://www.almazoptics.com/LiTaO3.html); we calculated that a sensor of 0.5 mm by 0.5 mm square would be able to produce a 0.1 mV change in potential when exposed to a 5·10⁻¹⁰° C. change in temperature. In other words, the PIR sensors would be able to detect even the most miniscule changes in blood temperature. A second question is if such changes in temperature are present in flowing blood. Earlier we indicated that there was a difference in ventilatory and heart rates as well as a pulsatile nature to blood flow. Not surprising then is that there are well-defined changes in temperature (FIG. 1). These temperature changes are orders of magnitude greater than the changes required for effective employment of PIR sensors.

The next aspect in the provision of an effective sensor system is to have a waveform maintain sufficient integrity from one PIR sensor to the next. In other words, having a temperature change observed by sensor 1 be observed by sensor 2. We placed two larger PIR sensors in a plastic fixture that encased a polyethylene tube (approximate diameter of 1 mm; FIG. 2). The sensors were connected to an oscilloscope. As is clearly evident in FIG. 2, a waveform that is detected by sensor 1 is also detectable by sensor 2. By exploiting Fourier transforms and waveform correlations of the corresponding signals, the blood flow rate between the two PIRs can be determined mathematically using the sensed data. The inventors have used Fourier transforms and waveform correlations to identify signal identity between sensors in previous projects.

FIG. 2 illustrates a demonstration of proof of concept. In the left panel, a polyethylene tube was encased in a fixture with two sensors a known distance apart. Water was circulated through this tube. A bolus of warm water was added. The peak corresponding to temperature (sensor potential on the Y axis) was detected initially at Sensor 1 and later at Sensor 2. Since we can calculate the time from peak to peak and we know the distance between the two sensors, a rate of flow can be determined for a given tube diameter. The system used for this demonstration is illustrated in the right panel. Note these are large sensors. It is believed that by using smaller sensors mounted more closely to the vessel, it is possible to decrease wavelength and better discern an individual peak. In the left panel, the x axis is the time and the y axis is PIR sensor potential which is directly related to sensed temperature. As the measurement of the temperature produces a definite time value across a specific length (the distance between the two locations of the sensors), and as the other parameters identified in the equations below are physically determinable (can be measured or are known), the rate of flow can be calculated from the sensed temperature data. The time required for the wave to travel from sensor 1 to sensor 2 is proportional to the rate of fluid flow. The diameter of the vessel and the distance between the two sensors is known) it can be measured or has already been measured by (for example) non-invasive visual determination by sonogram, X-ray, fluoroscopy, MRI and invasive measurements such as catheterization. Therefore, the rate of flow may be determined since flow rate=distance traveled/time and the volume of the tube between the sensors is calculated as V=πr²h where r is the radius (0.5×diameter) and h is the distance between the sensors. Therefore, the volume of fluid that is transported per unit time is calculable. This is the volumetric flow rate that is being determined from measurements from temperature sensing along the length of the vessel.

The next design feature that is desirable is to use a small detector for use with a blood vessel. While it is within the skill of the PhD level electrical engineer to build the proposed blood flow sensor from scratch, it would be better if a commercially available system was found and that components from off-the-shelf devices could be used to significantly reduce the sensor development time. Although PIR motion sensors have been around for decades, their miniaturization has only recently taken place to target the energy conservation market. Murata Electronics-North America now offers miniaturized PIR motion sensors with dimensions of 5.0×4.7×2.4 mm. This sensor is pre-packaged for surface mount applications and inappropriate for our needs. Fortunately, the sensor elements themselves are only 0.85×1.2 mm in dimensions, which is likely to be suitable for the proposed blood flow sensor. It is preferred that the sensors have dimensions of less than 1.0×1.2 mm, preferably less than 0.85×1.2 mm, and more preferably less than 0.7×1.0 mm, as with a range of from 0.2-1.0 mm (width)×0.4×1.2 mm (length) on the surface in contact with the vessel. In this project, we will use the sensor elements from these motion sensors, integrate them with the necessary electronics (e.g., field effect transistor), and mount them on an appropriate fixture for testing or implantation. The sensor elements are obtained directly from the vendor or remove them from pre-packaged motion sensor devices. We note that in FIG. 2, the sensor elements were removed from a pre-packaged motion sensor. The inventors' extensive experience with sputtering and making thin film materials was useful in this procedure. Should an issue of availability or suitability for our intended application arise, lithium tantalate is easily and readily applicable to a variety of surfaces (Denton et al., Denton R T, Chen F S, Ballman A A. (1967) Lithium tantalate light modulators. Journal of Applied Physics 38:1611-1617). In other words, we have the capacity and knowledge to manually construct our own sensors if warranted.

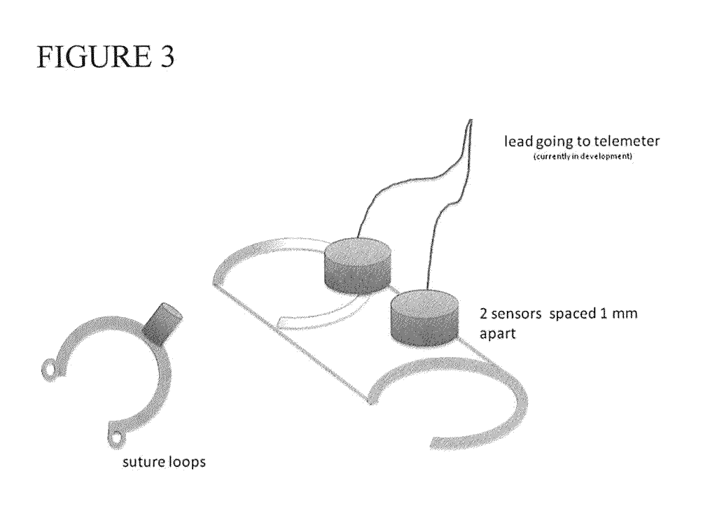

We anticipate further work using a thin but rigid plastic substrate to mount and immobilize the PIR elements. This mounting plate will then be encased in a flexible but moldable fixture for implantation on the blood vessel (see FIG. 3). Requirements for the substrate and fixture may include biocompatibility, electrical and thermal properties and suitability for component assembly. There are obvious substrates such as silicone based compounds that we will investigate and adapt for this project. We will use thin film interconnects for the electrical circuits as well as external connections for testing. Thin film interconnects are more appropriate for this application as they are smaller, more flexible and more reliable compared to wire interconnects. The inventors' laboratory routinely fabricates such circuits.

FIG. 3 illustrates an implantable mounting fixture. The device will be fitted around a blood vessel and tightened in place. Similar fixtures are currently used for vascular occluding.

Currently, there are very effective methods for measuring blood flow rate. A major goal of this sensor development was not to simply develop yet another method. Rather, we wanted a sensor that would consume no power and allow better integration to wireless telemetry. The present technology uses telemetry to monitor body temperature in hibernating ground squirrels. This provides background knowledge of competitive current technologies and approaches. The system would preferably embody a functional sensor using digital telemetry. This telemeter would allow long term and robust monitoring of blood flow.

The result of these practices is a novel and innovative approach to measuring blood flow rate that relies on very simple and proven technologies. This can provide a significant contribution to the realms of physiology and medicine with important consequences to the future development of therapies aimed at cardiac dysfunction and stroke.

The application of the PIR sensors is not limited to blood flow. It is reasonably extrapolated that the system should also be able to determine ventilatory rate (large inflections in FIG. 1) and heart rate (smaller inflections in FIG. 1) from the available data on blood temperature. Since blood pressure and temperature are correlated (Appelbaum et al., 1982) it is conceivable that we may should be able to determine blood pressure. Current ultrasonic flow methods with a calculated spectral broadening index are used to estimate severity and type of plaque formation due to increased turbulence in blood flow (Poepping et al., Poepping T L, Rankin R N, Holdsworth D W. (2010) Flow patterns in carotid bifurcation models using pulsed Doppler ultrasound: effect of concentric vs. eccentric stenosis on turbulence and recirculation. Ultrasound in Medicine and Biology 36:1125-1134.). We should be able to adapt our system with the addition of a downstream PIR sensor for a similar application. An important additional advantage of the proposed bold flow sensor is its low cost. The Murata miniature PIR sensor system sells for ˜$4 each (˜$2 each for quantities over 1,000).

What is claimed:

1. A method of determining the rate of flow of a bodily fluid through a vessel in a live patient comprising:
positioning at least two separate passive infrared sensors at two points along a length of the vessel;
sensing local temperature changes at the two points along the vessel to generate signals of the local temperature changes from each passive infrared motion detector;
a processor receiving the signals and quantifying the local temperature changes at the two points; and
the processor executing code to determine blood flow rate in the vessel from the local temperature changes quantified by the processor at the two points.

2. The method of claim 1 wherein motion is detected by at least two passive infrared sensors that comprise pyroelectric materials and the at least two passive infrared detectors produce an electrical potential.

3. The method of claim 2 wherein the pyroelectric material comprises lithium tantalite.

4. The method of claim 1 wherein each of the at least two passive infrared motions detectors has an area of contact with the vessel defined by 0.2-1.0 mm (width)×0.4×1.2 mm (length).

5. The method of claim 2 wherein each of the at least two passive infrared motions detectors has an area of contact with the vessel defined by 0.2-1.0 mm (width)×0.4×1.2 mm (length).

6. The method of claim 3 wherein each of the at least two passive infrared motions detectors has an area of contact with the vessel defined by 0.2-1.0 mm (width)×0.4×1.2 mm (length).

7. The method of claim 1 wherein the vessels are selected from the group consisting of veins and arteries.

8. The method of claim 5 wherein the vessels are selected from the group consisting of veins and arteries.

9. A system for measuring fluid flow in a vessel of a mammalian body, the system comprising:
at least two miniaturized passive infrared motion sensors, each sensor having a contact surface of between 0.2-1.0 mm (width)×0.4×1.2 mm (length);
each sensor being in communication link with a processor;
the processor configured to execute code converting signals from the sensors regarding temperature of fluid in the vessels to an indication of speed of fluid motion in the vessel.

10. The system of claim 9 wherein the at least two passive infrared sensors comprise pyroelectric materials.

11. The system of claim 10 wherein the at least two passive infrared sensors produce an electrical potential as a response to sensing temperature changes to generate a signal indicative of sensed temperature changes.

12. The method of claim 10 wherein the pyroelectric material comprises lithium tantalite.

13. The method of claim 11 wherein the pyroelectric material comprises lithium tantalite.
